(19) United States
(12) Patent Application Publication
(10) Pub. No.: US 2023/0260650 A1
(43) Pub. Date: Aug. 17, 2023

(54) Patient Analytics Directed to East Asian Medicine

(71) Applicant: Infinu Health, Inc., Austin, TX (US)

(72) Inventors: Simel Eric Osborne Jenkins Bey, Austin, TX (US); Jing Fan, Austin, TX (US); Zoltan Antal Papp, Spicewood, TX (US); Robert John Vanderwall, JR., Austin, TX (US)

(73) Assignee: Infinu Health, Inc., Austin, TX (US)

(21) Appl. No.: 17/672,875

(22) Filed: Feb. 16, 2022

(51) Int. Cl. G16H 50/20; G16H 50/70; G16H 10/60; G16H 20/90

(52) U.S. Cl. CPC G16H 50/20; G16H 50/70; G16H 10/60; G16H 20/90

(57) ABSTRACT

A system, method, and computer-readable medium are disclosed that provide patient analytics based East Asian Medicine (EAM) or Oriental Medicine (OM) or principles. Data specific to a patient along with environmental data related to the patient are collected. The collected data is processed using EAM principles through Artificial Intelligence/Machine Learning (AI/ML). The collected data is processed with EAM principles and output form AI/ML to provide patient health status over different time intervals. Diagnostics and recommendations are provided based on the AI/ML and symptoms checking.

### BACKGROUND OF THE INVENTION

### Field of the Invention

The present invention relates to the implementation of information handling systems directed to East Asian Medicine. More specifically, embodiments of the invention provide a system, method, and computer-readable medium for gathering data, performing machine learning and providing patient analytics based on East Asian Medicine or Oriental Medicine.

### Description of the Related Art

East Asian Medicine, also known as Oriental Medicine, is based on thousands of years of practice and theories. The practice of East Asian Medicine involves determining multivariate factors that are dynamic and ever-changing depending on time and environment. Practitioners rely on years of training, clinical practice, and accurate data collection and processing in determining the condition, diagnosis, and treatment of patients.

Because of the dynamic and ever changing nature of factors that affect a patient, and particularities specific to a patient, determining accurate and timely diagnostics and recommendations can be a challenge. To effectively provide accurate results, a skilled practitioner considers all related factors, determines relative environment and time, while typically performing manual calculations and diagnosis based on the practitioner's experience.

### SUMMARY OF THE INVENTION

A system, method, and computer-readable medium are disclosed for providing patient analytics based on East Asian Medicine (EAM) comprising collecting specific data related to a patient and environmental data related to the patient; processing the collected data with EAM principles through Artificial Intelligence/Machine Learning (AI/ML) to provide an AI/ML output; processing the collected data, EAM principles, and AI/ML output to provide symptoms checking for the patient that includes health states over different time intervals; and providing diagnostics and recommendations based on the AI/ML output and symptoms checking.

### BRIEF DESCRIPTION OF THE DRAWINGS

The present invention may be better understood, and its numerous objects, features and advantages made apparent to those skilled in the art by referencing the accompanying drawings. The use of the same reference number throughout the several figures designates a like or similar element.

FIG. 1 is a general illustration of components of an information handling system as implemented in the system and method of the present invention;

FIG. 2 is a simplified block diagram of an East Asian Medicine data gathering, machine learning, diagnostic, and recommendation system;

FIG. 3 is a block diagram of patient devices and bioinformatic sensors;

FIG. 4 shows screen presentations of graphical user interfaces for East Asian Medicine for data gathering, diagnostics and patient recommendations;

FIG. 5 illustrates examples of East Asian Medicine systems or principles;

FIG. 6 illustrates components of East Asian Medicine service;

FIG. 7 is a block diagram of interaction of components of East Asian Medicine service;

FIG. 8 is a screen presentation of a cancer predictor; and

FIG. 9 is a generalized flowchart for gathering data, performing machine learning and providing patient analytics based on East Asian Medicine.

### DETAILED DESCRIPTION

A system, method, and computer-readable medium are disclosed for gathering data, performing machine learning and providing patient analytics based on East Asian Medicine (EAM) or also known as Oriental Medicine (OM). The principles of EAM/OM are considered to provide medicinal diagnostics and recommendation based on space (environment) and time specific to a patient. Consideration is accounted for as to organ rate variability, and changes as to weather, health and climate. Various sources, including weather related sources, patient specific data (current and historical), patient demographic data, patient birthdate, etc. are provided as input to various machine learning engines. Implementations of the machine learning engines include convolutional neural networks.

For purposes of this disclosure, an information handling system may include any instrumentality or aggregate of instrumentalities operable to compute, classify, process, transmit, receive, retrieve, originate, switch, store, display, manifest, detect, record, reproduce, handle, or utilize any form of information, intelligence, or data for business, scientific, control, or other purposes. For example, an information handling system may be a mobile phone, personal computer, a network storage device, or any other suitable device and may vary in size, shape, performance, functionality, and price. The information handling system may include random access memory (RAM), one or more processing resources such as a central processing unit (CPU) or hardware or software control logic, ROM, and/or other types of nonvolatile memory. Additional components of the information handling system may include one or more disk drives, one or more network ports for communicating with external devices as well as various input and output (I/O) devices, such as a keyboard, a mouse, and a video display. The information handling system may also include one or more buses operable to transmit communications between the various hardware components.

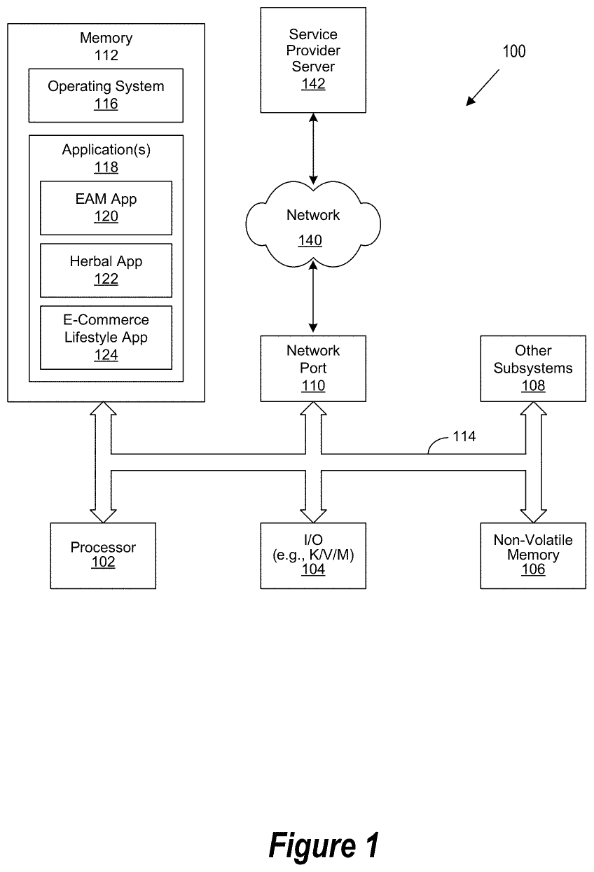

FIG. 1 is a generalized illustration of a user information handling system 100 that can be used to implement the system and method of the present invention. The information handling system 100 includes a processor (e.g., central processor unit or “CPU”) 102, input/output (I/O) devices 104, such as a keyboard, a video/display, a mouse, and associated controllers (e.g., K/V/M), a hard drive or disk storage 106, and various other subsystems 108. In various embodiments, the information handling system 100 also includes network port 110 operable to connect to a network 140, which is likewise accessible by a service provider server 142. The information handling system 100 likewise includes system memory 112, which is interconnected to the foregoing via one or more buses 114. System memory 112 further comprises operating system (OS) 116 and in various embodiments may also include application(s) 118 that are configured to perform various operations and tasks on the information handling system 100.

Various implementations provide for applications 118 to include an OM application 120, an herbal application 122 and an e-commerce lifestyle application 124 which interact with other elements as further described here.

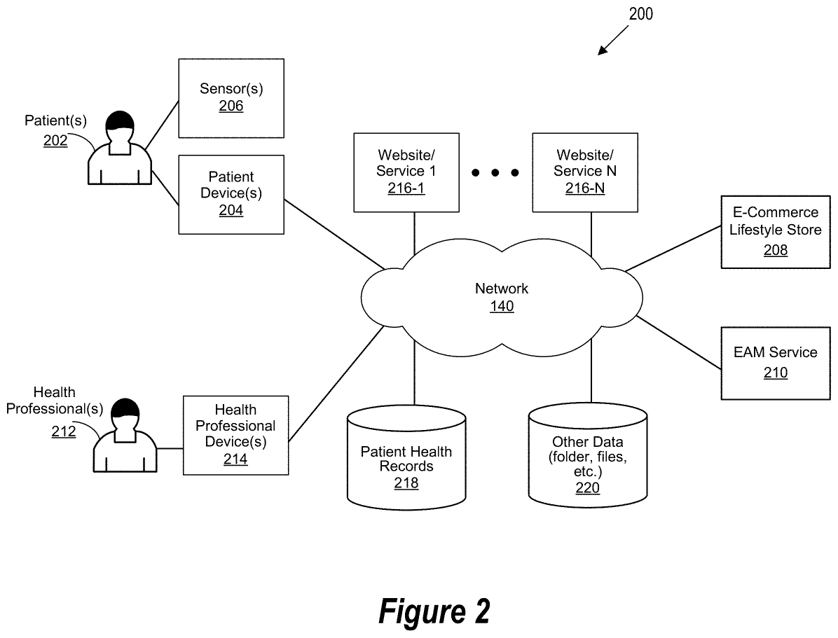

FIG. 2 is a simplified block diagram of an East Asian Medicine (EAM) data gathering, machine learning, diagnostic, and recommendation system 200. In various embodiments, the system 200 provides for patient(s) 202 through patient devices 204 to connect to the network 140.

The network 140 may be a public network, such as the Internet, a physical private network, a wireless/wired network, a virtual private network (VPN), or any combination thereof. For example, the network 140 can include 5G wireless networks, and future evolutions of 5G. 5G and future standards can provide for the ability to seamlessly connect devices, such as patient devices 200 and embedded sensors in and connected to patient devices 200, and can scale down in data rates, power, and mobility providing efficient and low-cost connectivity solutions. Skilled practitioners of the art will recognize that many such embodiments are possible, and the foregoing is not intended to limit the spirit, scope or intent of the invention.

Patient(s) 202 are representative of individuals at various stage in the life cycle. The life cycle includes birth, youth, teen, young adult, adult, middle age, gerontology and death. Patient(s) 202 are unique and have specific data and records. Patient(s) include both human and animals. Animals can be pets and livestock.

The patient devices 204 can refer to an information handling system 100 as described in FIG. 1, and can include for example as a personal computer, a laptop computer, a tablet computer, a personal digital assistant (PDA), a smart phone, a mobile telephone, or other device that is capable of communicating and processing data. As commonly known, patient devices 204 can be considered as Internet of Things (IoT) that includes physical devices (or groups of devices) that can include or access sensors, with the processing capabilities and software to connect and exchange data with other devices and system over the network 140 including the Internet.

Various implementation provide for patient(s) 202 and patient device(s) 204 to connect to various sensor(s) 206. Sensor(s) 206 can include patient wearable devices, such as patches, watches, etc. that are configured to monitor and gather patient 202 data. Such patient sensor(s) 206 are further described herein. Furthermore, sensor(s) 206 can include environmental sensors that monitor and gather environmental data experienced at patient device(s) 204. Such environmental sensor(s) 206 can be included in patient device(s) 204.

Implementations provide for the system 200 to include an e-commerce lifestyle store 208, which can be a website(s) accessed through network 140. As described above, information handling system 100 can include an e-commerce lifestyle application 124. The e-commerce lifestyle application 124 can be used by patient(s) 202 to access the e-commerce lifestyle store 208 for goods and/or services.

Implementations provide for the system 200 to include an East Asian Medicine (EAM) service 210, which can be a web site(s) accessed through network 140. As described above, information handling system 100 can include an EAM application 120. The EAM application 120 can be used by patient(s) 202 to access the EAM service 210, for services such as diagnostics and recommendations related to health care based on EAM principles. The EAM service 210 is further described herein.

The system 200 can provide access to elements of system 200, by health professional(s) 212 through health professional device(s) 214. The health professional device(s) 214 access other elements of system 200 through network 140. Implementations provide for the health professional device(s) 214 to be embodied as an information handling system 100 as described in FIG. 1.

The system 200 can further include one or more websites or services as represented by website/service 216-1 to 216-N. Examples of website/service 216 include real time weather or environmental websites or services, world clock websites, and other data sites. Examples of websites 216 include National Oceanic and Atmospheric Administration (NOAA) website, weather.com website, etc.

A principle of EAM is “Wu Yin Liu Qi,” which has five components of weather, earth, plants, animals, and human. The five components can be considered as Internet of Medical Things (IoMT). Weather/weather phenomena are studied and correlated to heath of patient(s) 202. Changing weather, weather patterns as related to earth, space and relationship to health of patient(s) 202 is a principle of “Wu Yin Liu Qi.” “Wu Yin” can be interpreted as relating to change of time, direction, etc. of five seasons of spring, summer, late summer, fall and winter. “Liu Qi” can be interpreted as to six kids of climate change: wind, dampness, summer-heat, fire-heat, dryness, and cold.

Data from weather and climate related websites/services 222 correlate with location of patient(s) 202, and particularly patient device(s) 204. Such data can be really time data and can include time stamps indicating the time such data is collected/gathered. Implementations provide for the various elements of system 200 to access websites/services 222 and use the data for the methods described herein.

Implementations provide for the system 200 to include patient health records database(s) 218, and other data database(s) 220. The patient health records database 218 can be accessed and/or written to by elements of system 200. In particular, patient device(s) 204, health professional device(s) 214, and EAM service 210 can access and/or write to patient health records database(s) 218. Other data database(s) 220 can include data folders, files, etc. which include gathered and processed data by elements of system 200 as to patient(s) 202.

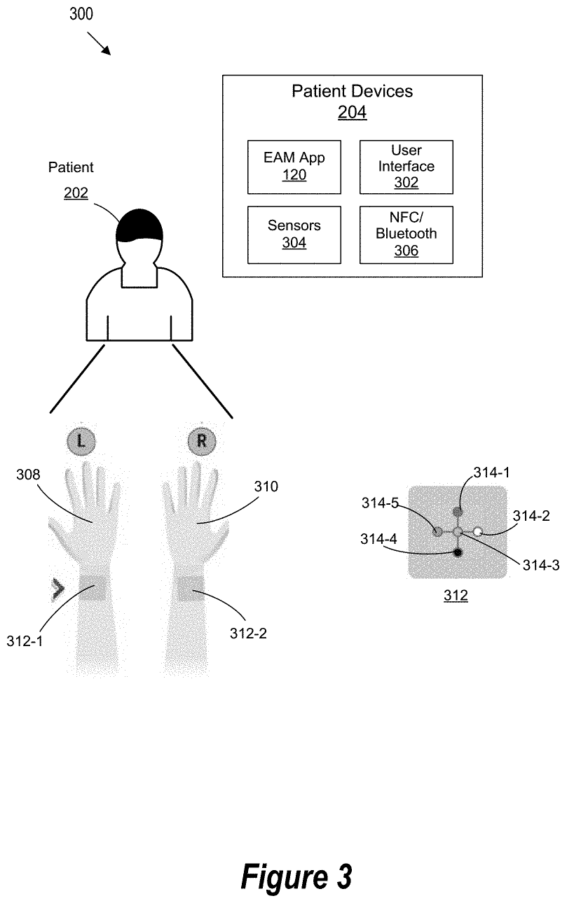

FIG. 3 shows a block diagram of an example configuration and interaction 300 of a patient 202 with patient devices 204 and bioinformatic sensors, such as sensor(s) 206. A patient 202 interacts with a patient device 204. Implementations provide for patient device 204 to include the EAM app 120 and a user interface 302. The user interface can be used by patient 202 to enter data and information. The user interface 302 can also provide for data and information to be provided to patient 202, such as outcome, diagnostic, recommendation, cancer prediction, etc. data and information.

Implementations also provide for the patient devices 204 to include sensors 304 (e.g., environmental sensors 206), which are configured to monitor environmental conditions, such as humidity, temperature, etc. which are used to derive outcomes in EAM. Implementations can include for the patient devices 204 to include communications such as NFC/Bluetooth 306. In particular, such communications can be used to receive data from bioinformatic sensors (e.g., sensors 206) of the patient 202.

In certain implementations, left hand 308 and right hand 310 of patient 202 include a radial (on radial artery) patch 312-1 and a radial patch 312-2. Implementations provide for either one or two patches 312. Patch 312 can measure the radial pulse of patient 202 as well as temperature, skin moisture/humidity, sweat, oxygenation, etc. EAM relies on what is referred to as the four humors related to heat cold, dryness, and dampness. Patches 312 are configured to gather patient specific data as to the four humors. The data can be gathered in real time and time correlated. EAC relates organ rate variability or how organs of the patient 202 are affected with variability in the four humors.

Implementations provide for the patches 312 to operate in a time based series to track potential health risk patterns in real time using temperature and organ rate variability. Dynamic organ health variability is a time based series that is determinative of organs such as the heart, small intestine, bladder, kidney, pericardium, three warmers, gall bladder, liver, lungs, large intestine, stomach, and spleen. The left and right side of the patient 202 may be specific to particular organs. Therefore, two patches 312 may be implemented for particular organs; however, it is to be understood that one patch 312 may be implemented.

The patch 312 can include one or more sensors 314. Each sensor 314 can be configured to perform a particular measurement, such temperature, oxygenation, pulse rate, dampness/dryness, etc. For example, the data from the sensors 314 can be received by patient devices 204 through NFC/Bluetooth 306. Patches 312 are one implementation of a wearable device. Other wearable devices can be implemented to gather data as described herein. In various implementations, the use of such wearable devices is used with in medical interviews with health professional(s) 212.

The human body is considered to have 12 systems consisting of the skeletal system, the nervous system, the muscular system, the respiratory system, the endocrine system, the immune system, the cardiovascular system, the circulatory system, the urinary system, the integumentary system, the reproductive system, and the digestive system. Implementations provide for wearable devices, such as patches 312, to be configured to remotely monitor the 12 systems related to temperature, cold, heat, dampness, heart rate variability, and organ rate variability. Various implementations provide for data from wearable devices to be provided to a health engine as further described herein.

EAM principles include body maps and graph zones describing the condition of patient(s) 202. Implementations provide for the wearable devices to provide data to a knowledge base server, provide dynamic health category of concern detected in real time on a dynamic body map, provide dynamic health score populated by customer changes (spirit, mind, body) lifestyle, and provided curated content based upon preferences.

Implementations provide for wearable devices to be used with herbal application 122 and e-commerce lifestyle store 208. Wearable devices provide input/data, and particularly content source identity from curated content and entrance to with herbal application 122 and e-commerce lifestyle store 208. Data from wearable devices can also be stored and found in other data database(s) 220, particularly folders and files.

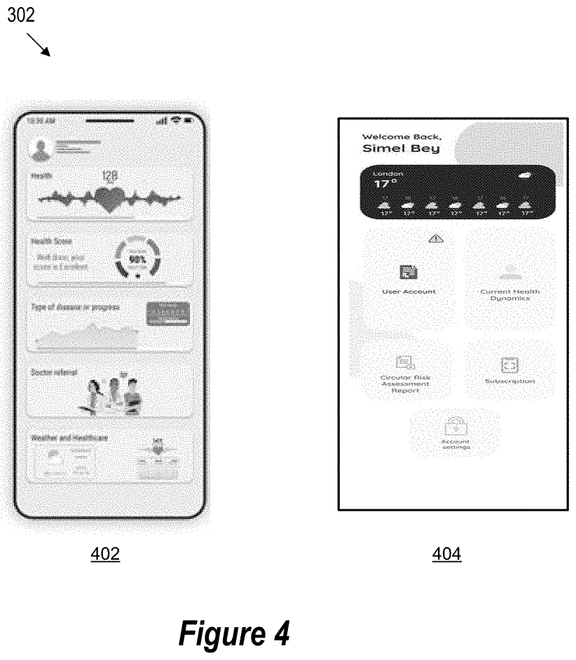

FIG. 4 shows example screen presentations of graphical user interfaces (GUI) for EAM data gathering, diagnostics, and patient recommendations. The user interface 302 can include GUI 402 and GUI 404. As an example, GUI 402 can be used for entering information by patient(s) 202 and for receiving information through patient device(s) 204. GUI 404 is an example user interface that can be used with weather health engine further describe herein, that predicts health based on dynamic weather conditions/changes.

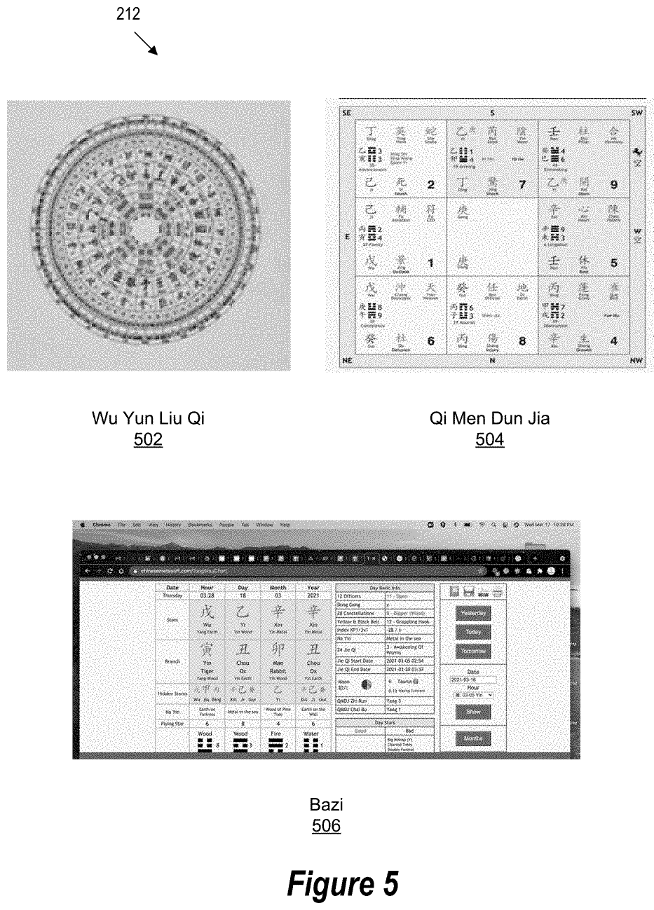

FIG. 5 shows examples of EAM systems/principles 500. EAM theory can include the following theory: four stages of Yin Yang (excess/deficiency); five phases that include wood, fire, metal and water; eight rubrics (diagnostics); ten stems (celestial phenomena changes); twelve branches (earth time stamp changes); twelve organs and channels; twenty four jie qi (predictable periodic earth changes); twenty eight constellations; sixty cycles and sixty four hexagrams.

In particular, the EAM systems/principles 500 are used to create and detect dynamic health changes. Examples of EAM systems/principles 500 can include the Wu Yun Liu Qi 502, Qi Men Dun Jia (four mystic doors) 504, and Bazi (four pillars or eight characters) 506. It is to be understood that other EAM systems or principles can be implemented as part of EAM systems/principles 500.

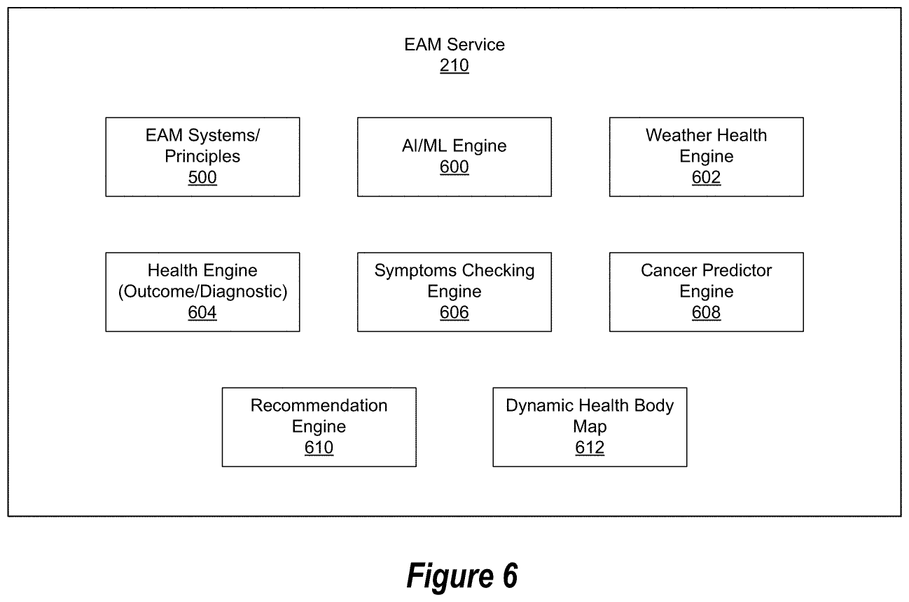

FIG. 6 shows components of a EAM service 210. As described, implementations provide for patient devices 204 to connect with EAM service 210. In particular, implementations provide for EAM application 120 to connect with an receive data and information from EAM service 210. Embodiments include EAM service 210 can be provided by one or more computing devices, such as server computers. Embodiments can also provide that EAM service 210 to be implemented in a cloud computing environment.

Embodiments provide for the EAM service 210 to include EAM systems/principles 500. The EAM systems/principles 500 can be implemented in one or more databases (e.g., other data database(s) 220) as part of or accessible by the EAM service 210. Implementations provide for the EAM service 210 to include an artificial intelligence/machine learning engine 600. The artificial intelligence/machine learning engine 600 can be implemented to receive data and information as further described herein and refine and provide for more accurate input that is used by other engines and applications of the EAM service 210.

Implementations provide for the EAM service 210 to include a weather health engine 602. Weather health engine 602 is implemented to combine dynamic health changes according to weather patterns, such as received from data sources such as online websites/services 216. Such health weather application receives results from OM service 208 and can integrate with the system checking engine 214, patch 312, and cancer predictor engine 220. The weather health engine 602 detects adverse weather patterns that can affect patient(s) 202 health and provide predictions as to changes in dynamic health conditions. In addition, implementations provide for the Weather health engine 602 to provide curated content as predictive, preventative, and prescriptive recommendations.

In general, the weather health engine 602 determines effects (interior) to the patient(s) 202. The weather health engine 602 identifies with sky, humans, animals, soil, botany/plants, insects. The weather health engine 602 provides data for EAM graph zones as to weather and temperature. EAM graph zones are provided data from website 216 data points (e.g., NOAA) translated for human, animals, plants and insects. A dynamic health score can be provided based on changes upon the hour, day, week, month, year, geolocation, lifestyle, etc. A dynamic health category can be flagged if there is a concern.

Implementations provide for weather health engine 602 to interact with herbal application 122 and e-commerce lifestyle store 208. Weather health engine 602 can provide input/data, and particularly content source identity from curated content and entrance to with herbal application 122 and e-commerce lifestyle store 208. Data from weather health engine 602 can also be stored and found in other data database(s) 220, particularly folders and files.

Implementations provide for the EAM service 210 to include a health engine (outcome/diagnostics) 604. In various implementations the health engine 604 provides an action to support health professional(s) 212 to conduct private medical interviews (PMI) with patient(s) 202. Patient(s) 202 can be identified as to name, birth date, address, country, medical history, current medical history, medical disclaimer, various questions, etc.

The health engine 604 provides data for EAM graph zones as to an inquiry process: observation, listening, palpation, smelling, taste, etc. which can be provided to a knowledge based server. A dynamic health score can be provided. A dynamic health category can be flagged if there is a concern. A dynamic circular biomedicine risks assessment of the 12 systems can be performed as to EAM risk. Curated content can be performed based upon preferences and assessments.

Implementations provide for health engine 604 to interact with herbal application 122 and e-commerce lifestyle store 208. Health engine 604 can provide input/data, and particularly content source identity from curated content. Data from health engine 604 can also be stored and found in other data database(s) 220, particularly folders and files.

Implementations provide for the EAM service 210 to include a symptoms checking engine 606, an outcome/diagnostic engine 608, a recommendation engine 610 and cancer predictor engine 612 as further described herein. Implementations provide for the symptoms checking engine 606 to be a whole health system checker that predicts curated content for probable disease/health states based on different time intervals, such as hourly, daily, weekly, monthly and annually.

Implementations provide for the EAM service 210 to include and provide for a patient dynamic health body map 612. The dynamic health body map 612 can provide real time changes. In various implementations, the dynamic health body map 612 is a multidimensional clickable thermographic (human) body graph that provides for surface to bone diagnosis of the 12 biomedical systems, and EAM meridian channel systems diagnosis. Furthermore, the patient(s) 202 are matched as to real time height, weight, etc., when health professional(s) 212 conduct private medical interviews (PMI) with patient(s) 202. The dynamic health body map 612 can be share data with other systems and libraries and can change as to weather.

The dynamic health body map 612 provides data for EAM graph zones as to an inquiry process: observation, listening, palpation, smelling, taste, etc. which can be provided to a knowledge based server. Photographs or images of the tongue, eye and face can be mapped for each zone as to EAM. A dynamic health score can be provided. A dynamic health category can be flagged if there is a concern on the dynamic health body map 612. A dynamic health score can be automatically populated by patient(s) 202 per questions answered. Curated content can be performed based upon references.

Implementations provide for dynamic health body map 612 to interact with herbal application 122 and e-commerce lifestyle store 208. Dynamic health body map 612 can provide input/data, and particularly content source identity from curated content and entrance to with herbal application 122 and e-commerce lifestyle store 208. Data from dynamic health body map 612 can also be stored and found in other data database(s) 220, particularly folders and files.

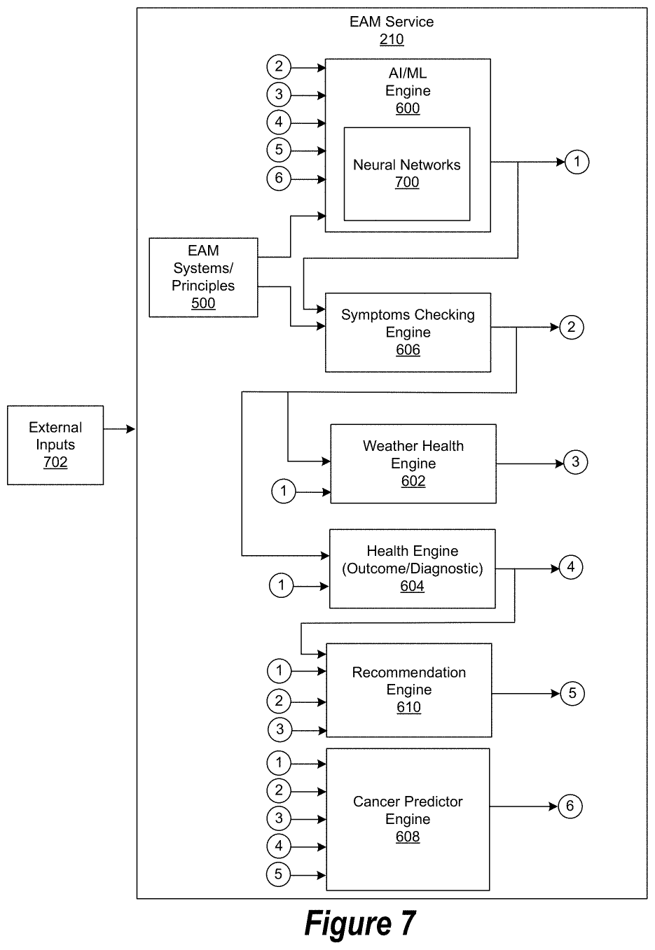

FIG. 7 shows an interaction of components of EAM service 210. In various embodiments the artificial intelligence/machine learning engine 600 includes one or more artificial neural networks 700. Generally, neural networks 700 are used to solve or refine answers to artificial intelligence or machine learning problems, and specifically performing health related problems or issues related to EAM. The neural networks 700 can include an input layer, middle/hidden layer, and output layer. Furthermore, neural networks 700 can be one of various types of neural networks, such as recurrent neural networks, concurrent neural networks, etc.

Embodiments provide for the EAM service 210 and components to receive external inputs 702, which include as described above, data and information for patient devices 204, websites/services 216, patient health records 218, other data database(s) 220 and health professional devices 214.

Embodiments provide for artificial intelligence/machine learning engine 600 to received external inputs, and input from EAM systems/principles 500, symptoms checking engine 606, weather health engine 602, health engine (outcome/diagnostic) 604, recommendation engine 610, and cancer predictor engine 608. Implementation can provide for neural networks 700 to receive the inputs in an input layer, process the inputs in a hidden layer and provide results in an output layer.

Implementations can include that the symptoms checking engine 606 to receive inputs from the artificial intelligence/machine learning engine 210 and EAM systems/principles 500 and provide for an output. As discussed, implementations can provide for the symptoms checking engine 606 to be a whole health system checker that predicts curated content for probable disease/health states based on different time intervals, such as hourly, daily, weekly, monthly and annually.

Implementations provide for the weather health engine 602 to receive inputs from the artificial intelligence/machine learning engine 600 and symptoms checking engine 606 and provide for an output.

Implementations provide for the health engine (outcome/diagnostic) 604 to receive inputs from the artificial intelligence/machine learning engine 600 and symptoms checking engine 606 and provide for an output.

Implementations can provide for recommendation engine 610 to receive inputs from the artificial intelligence/machine learning engine 600, symptoms checking engine 606, weather health engine 602, and health engine (outcome/diagnostic) 604, and provide for an output.

The cancer predictor engine 608 can receive inputs from artificial intelligence/machine learning engine 600, symptoms checking engine 606, weather health engine 602, health engine (outcome/diagnostic) 604, and recommendation engine 610. The cancer predictor engine 608 relies on the dynamic changes to the health of a patient(s) 202 based on the principles of EAM. Therefore, the outputs of the respective components are determinative of cancer predictor engine 608 predictions.

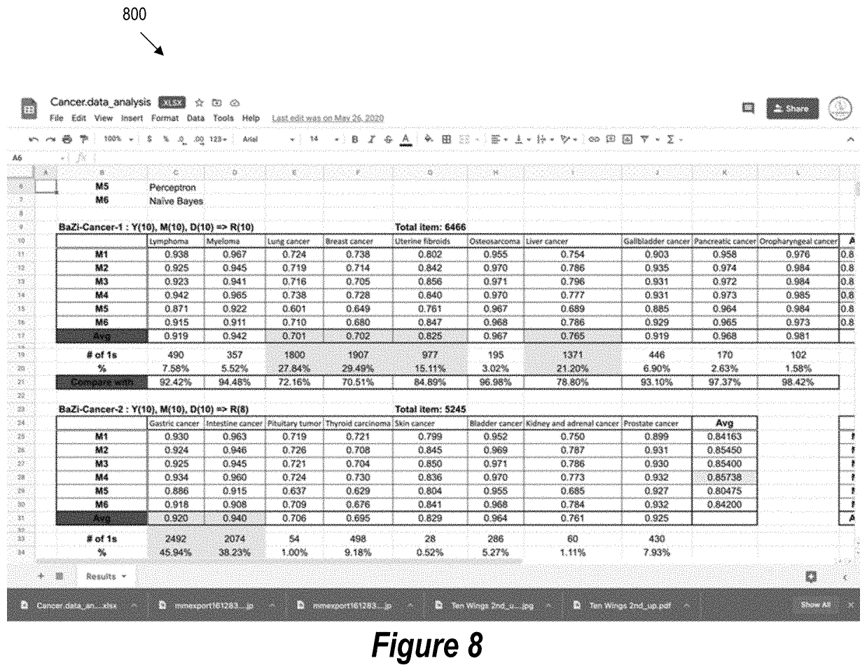

FIG. 8 shows an example screen presentation of a prediction from the cancer predictor engine 220.

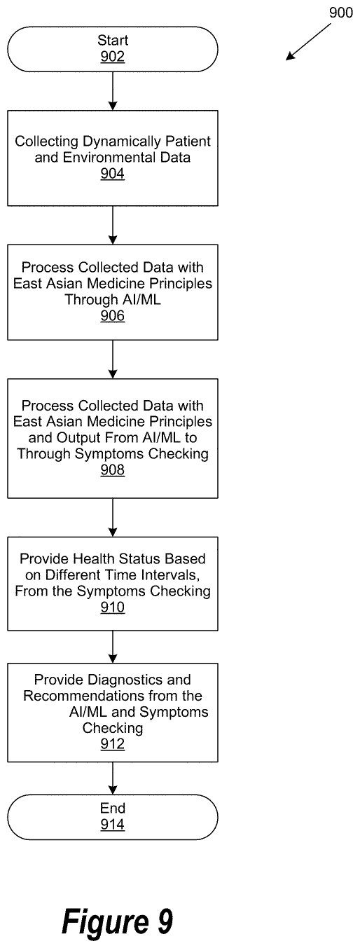

FIG. 9 is a generalized flowchart 900 for gathering data, performing machine learning and providing patient analytics based on East Asian Medicine. In various embodiments, the system 200 is implemented. The order in which the method is described is not intended to be construed as a limitation, and any number of the described method blocks may be combined in any order to implement the method, or alternate method. Additionally, individual blocks may be deleted from the method without departing from the spirit and scope of the subject matter described herein. Furthermore, the method may be implemented in any suitable hardware, software, firmware, or a combination thereof, without departing from the scope of the invention.

At block 902, process 900 starts. At step 904, patient and environmental data related to the data are collected. The collection can be performed dynamically over a period of time (i.e., time based or time variant). At step 906, using artificial intelligence/machine learning, and principles of EAM the collected data is processed. At step 908, the collected data, principles of EAM and output from artificial intelligence/machine learning are processed through symptoms checking. At step 910, health or disease status is provided based on the output processed by symptoms checking. Health or disease status can be based on different time intervals. At step 912, diagnostics based on outputs from that artificial intelligence/machine learning and symptoms checking are provided. At step 914, the process 900 ends.

The present invention is well adapted to attain the advantages mentioned as well as others inherent therein. While the present invention has been depicted, described, and is defined by reference to particular embodiments of the invention, such references do not imply a limitation on the invention, and no such limitation is to be inferred. The invention is capable of considerable modification, alteration, and equivalents in form and function, as will occur to those ordinarily skilled in the pertinent arts. The depicted and described embodiments are examples only and are not exhaustive of the scope of the invention.

As will be appreciated by one skilled in the art, the present invention may be embodied as a method, system, or computer program product. Accordingly, embodiments of the invention may be implemented entirely in hardware, entirely in software (including firmware, resident software, micro-code, etc.) or in an embodiment combining software and hardware. These various embodiments may all generally be referred to herein as a “circuit,” “module,” or “system.” Furthermore, the present invention may take the form of a computer program product on a computer-usable storage medium having computer-usable program code embodied in the medium.

Any suitable computer usable or computer readable medium may be utilized. The computer-usable or computer-readable medium may be, for example, but not limited to, an electronic, magnetic, optical, electromagnetic, infrared, or semiconductor system, apparatus, or device. More specific examples (a non-exhaustive list) of the computer-readable medium would include the following: a portable computer diskette, a hard disk, a random access memory (RAM), a read-only memory (ROM), an erasable programmable read-only memory (EPROM or Flash memory), a portable compact disc read-only memory (CD-ROM), an optical storage device, or a magnetic storage device. In the context of this document, a computer-usable or computer-readable medium may be any medium that can contain, store, communicate, or transport the program for use by or in connection with the instruction execution system, apparatus, or device.

Computer program code for carrying out operations of the present invention may be written in an object oriented programming language such as Java, Smalltalk, C++ or the like. However, the computer program code for carrying out operations of the present invention may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network (LAN) or a wide area network (WAN), or the connection may be made to an external computer (for example, through the Internet using an Internet Service Provider).

Embodiments of the invention are described with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The present invention is well adapted to attain the advantages mentioned as well as others inherent therein. While the present invention has been depicted, described, and is defined by reference to particular embodiments of the invention, such references do not imply a limitation on the invention, and no such limitation is to be inferred. The invention is capable of considerable modification, alteration, and equivalents in form and function, as will occur to those ordinarily skilled in the pertinent arts. The depicted and described embodiments are examples only and are not exhaustive of the scope of the invention.

Consequently, the invention is intended to be limited only by the spirit and scope of the appended claims, giving full cognizance to equivalents in all respects

What is claimed is:

1. A computer-implementable method for providing patient analytics based on East Asian Medicine (EAM) comprising:
collecting specific data related to a patient and environmental data related to the patient;
processing the collected data with EAM principles through Artificial Intelligence/Machine Learning (AI/ML) to provide an AI/ML output;
processing the collected data, EAM principles, and AI/ML output to provide symptoms checking for the patient that includes health states over different time intervals; and
providing diagnostics and recommendations based on the AI/ML output and symptoms checking.

2. The method of claim 1, wherein the patient data is gathered from patient devices and/or patient health records.

3. The method of claim 1, wherein the environmental data is gathered from sensors of a patient device and/or websites/services.

4. The method of claim 1, wherein a bioinformatic sensor is configured to gather patient data.

5. The method of claim 1, wherein the health states are related to weather conditions.

6. The method of claim 1, wherein the AI/ML includes one or more neural networks.

7. The method of claim 1 further comprising predicting cancer state included in the health states over different time intervals.

8. A system comprising:
a processor;
a data bus coupled to the processor; and
a non-transitory, computer-readable storage medium embodying computer program code, the non-transitory, computer-readable storage medium being coupled to the data bus, the computer program code interacting with a plurality of computer operations and comprising instructions executable by the processor and configured for:
collecting specific data related to a patient and environmental data related to the patient;
processing the collected data with East Asian Medicine (EAM) principles through Artificial Intelligence/Machine Learning (AI/ML) to provide an AI/ML output;
processing the collected data, EAM principles, and AI/ML output to provide symptoms checking for the patient that includes health states over different time intervals; and
providing diagnostics and recommendations based on the AI/ML output and symptoms checking.

9. The system of claim 8, wherein the patient data is gathered from patient devices and/or patient health records.

10. The system of claim 8, wherein the environmental data is gathered from sensors of a patient device and/or websites/services.

11. The system of claim 8, wherein a bioinformatic sensor is configured to gather patient data.

12. The system of claim 8, wherein the health states are related to weather conditions.

13. The system of claim 8, wherein the AI/ML includes one or more neural networks.

14. The system of claim 8 further comprising predicting cancer state included in the health states over different time intervals.

15. A non-transitory, computer-readable storage medium embodying computer program code, the computer program code comprising computer executable instructions configured for:
collecting specific data related to a patient and environmental data related to the patient;
processing the collected data with East Asian Medicine (EAM) principles through Artificial Intelligence/Machine Learning (AI/ML) to provide an AI/ML output;
processing the collected data, EAM principles, and AI/ML output to provide symptoms checking for the patient that includes health states over different time intervals; and
providing diagnostics and recommendations based on the AI/ML output and symptoms checking.

16. The non-transitory, computer-readable storage medium of claim 15, wherein the patient data is gathered from patient devices and/or patient health records, and the environmental data is gathered from sensors of a patient device and/or web sites/services.

17. The non-transitory, computer-readable storage medium of claim 15, wherein a bioinformatic sensor is configured to gather patient data.

18. The non-transitory, computer-readable storage medium of claim 15, wherein the health states are related to weather conditions.

19. The non-transitory, computer-readable storage medium of claim 15, wherein the AI/ML includes one or more neural networks.

20. The non-transitory, computer-readable storage medium of claim 15 further comprising predicting cancer state included in the health states over different time intervals.
